(19) United States
(12) Patent Application Publication
(10) Pub. No.: US 2005/0191344 A1
(43) Pub. Date: Sep. 1, 2005

(54) Liposome composition for delivery of therapeutic agents

(76) Inventors: Samuel Zalipsky, Redwood City, CA (US); Weiming Zhang, San Francisco, CA (US); Kew Shi Kun Huang, Castro Valley, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/036,523

(22) Filed: Jan. 13, 2005

Related U.S. Application Data
(60) Provisional application No. 60/513,864, filed on Jan. 15, 2004.

(51) Int. Cl. A61K 48/00; A61K 9/127

(52) U.S. Cl. 424/450; 554/78; 548/112; 514/44

(57) ABSTRACT

A neutral cationic lipid and liposomes prepared from the neutral cationic lipid are described. Liposomes comprised of the lipid are suitable for delivery of a polyanionic compound, such as a nucleic acid. The delivery can be performed in vivo or ex vivo. The neutral cationic lipid, which is neutral in charge at physiologic pH and positively charged at pH values less than physiologic pH, contains a polar head group that imparts solubility of the lipid and permits its packing into a liposomal lipid bilayer.

This application claims the benefit of U.S. Provisional Application No. 60/513,864, filed Jan. 15, 2004, incorporated herein by reference in its entirety.

### FIELD OF THE INVENTION

The present invention relates to liposome compositions for delivery of therapeutic agents, polyanionic compounds in particular, and especially nucleic acids. More particularly, the invention relates to a liposome composition that includes a weakly cationic lipid and optionally a surface coating of hydrophilic polymer chains and/or a targeting ligand for use in in vivo or ex vivo delivery of therapeutic agents, including polyanionic compounds such as polynucleotides.

### BACKGROUND OF THE INVENTION

A variety of methods have been developed to facilitate the transfer of genetic material into specific cells. These methods are useful for both in vivo or ex vivo gene transfer. In the former, a gene is directly introduced (intravenously, intraperitoneally, aerosol, etc.) into a subject. In ex vivo (or in vitro) gene transfer, the gene is introduced into cells after removal of the cells from specific tissue of an individual. The transfected cells are then introduced back into the subject.

Delivery systems for achieving in vivo and ex vivo gene therapy include viral vectors, such as retroviral vectors or adenovirus vectors, microinjection, electroporation, protoplast fusion, calcium phosphate, and liposomes (Felgner, J., et al., Proc. Natl. Acad. Sci. USA 84:7413-7417 (1987); Mulligan, R. S., Science 260:926-932 (1993); Morishita, R., et al., J. Clin. Invest. 91:2580-2585 (1993)).

The use of cationic lipids, e.g., derivatives of lipids with a positively charged ammonium or sulfonium ion-containing headgroup, for delivery of negatively-charged biomolecules, such as oligonucleotides and DNA fragments, as a liposome lipid bilayer component is widely reported. The positively-charged headgroup of the lipid interacts with the negatively-charged cell surface, facilitating contact and delivery of the biomolecule to the cell. The positive charge of the cationic lipid is further important for nucleic acid complexation.

However, systemic administration of such cationic liposome/nucleic acid complexes leads to their facile entrapment in the lung. This lung localization is caused by the strong positive surface charge of the conventional cationic complexes. In vivo gene expression of the conventional cationic complexes with reporter gene has been documented in the lung, heart, liver, kidney, and spleen following intravenous administration. However, morphological examination indicates that the majority of the expression is in endothelial cells lining the blood vessels in the lung. A potential explanation for this observation is that the lung is the first organ that cationic liposome/nucleic acid complexes encounter after intravenous injection. Additionally, there is a large surface area of endothelial cells in the lung, which provides a readily accessible target for the cationic liposome/nucleic acid complexes.

Although early results were encouraging, intravenous injection of simple cationic liposomes has not proved useful for the delivery of genes to systemic sites of disease (such as solid tumors other than lung tumors) or to the desired sites for clinically relevant gene expression (such as p53 or HSV-tk). Cationic liposomes are cleared too rapidly, and present a host of safety concerns. For example, Senior et al. (Biochim. Biophys. Acta 1070, 173-179 (1991)) reported that stearylamine containing liposomes interacted in a charge and concentration dependent manner with plasma and isolated erythrocytes. Gross interactions were observed between plasma components and erythrocytes, including formation of clot-like masses and hemolysis of erythrocytes, suggesting rapid clearance in vivo and trapping of liposomes in lung capillaries.

Furthermore, Filion et al. (Filion, M. C. and Phillips, N. C. (1998) Int. J. Pharmaceutics 162: 159-170) reported that cationic liposomes pose a risk of toxicity to phagocytic cells such as macrophages. Incubation of macrophages with cationic liposomes in vitro under non toxic conditions or in vivo resulted in the down-regulation of the synthesis of the protein kinase C dependent mediators nitric acid, tumor necrosis factor-α and prostaglandin E₂ by activated macrophages. Exposure of macrophages to cationic liposomes for times in excess of 3 hours resulted in a high level of toxicity (ED₅₀<50 nmol/ml).

An alternative to the use of cationic liposomes has been to include in the liposome a pH sensitive lipid, such as palmitoylhomocysteine (Connor, J., et al., Proc. Natl. Acad. Sci. USA 81:1715 (1984); Chu, C.-J. and Szoka, F., J. Liposome Res. 4(1):361 (1994)). Such pH sensitive lipids at neutral pH are negatively charged and are stably incorporated into the liposome lipid bilayers. However, at weakly acidic pH (pH<6.8) the lipid becomes neutral in charge and changes in structure sufficiently to destabilize the liposome bilayers. The lipid when incorporated into a liposome that has been taken into an endosome, where the pH is reported to be between 5.0-6.0, destabilizes and causes a release of the liposome contents.

Another approach has been to incorporate neutral cationic lipids into liposomes for delivery of associated agents, such as nucleic acids. As described in U.S. Patent Application Publication No.: U.S. 2003/0031764, such liposomes possess a reduced surface charge at physiological pH, and thus are less likely to become entrapped in the lung or other organs. However, the lipids described in the aforementioned patent application lack a polar headgroup which can lead to reduced solubility in some solvents.

In addition, tumor cell direct targeting is much more challenging than angiogenic endothelial cell targeting. Liposome/DNA complexes access angiogenic endothelial cells of tumor vasculature relatively easily, since the cells are directly exposed in the blood compartment. For targeting of tumor cells, liposome/DNA complexes need to be able to extravasate through the leaky tumor blood vessels to reach tumor cells. Thus the complex stability, size, surface charge, blood circulation time, and transfection efficiency of complexes are all factors for tumor cell transfection and expression.

### SUMMARY OF THE INVENTION

Accordingly, it is an object of the invention to provide a composition for systemic delivery of polyanionic compounds, such as nucleic acids, to a cell.

It is another object of the invention to provide a liposome comprising a neutral cationic lipid, wherein the liposome is associated with a nucleic acid for subsequent delivery of the nucleic acid to a cell or tissue.

It is yet another object of the invention to provide a liposome comprising a lipid derivatized with a hydrophilic polymer.

It is yet another object of the invention to provide a liposome composition for gene delivery or genetic modulation in a target tissue or cell, the liposome having an extended circulation time in the patient's blood.

Accordingly, in one aspect, the invention includes a composition for administration of a polyanionic compound, comprising:
liposomes comprising
   (i) a neutral cationic lipid having a structure according to formula (I) 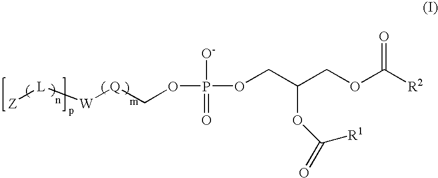
      wherein each of R¹ and R² is a branched or unbranched alkyl, alkenyl or alkynyl chain having between 6-24 carbon atoms;
   n=1-20;
   m=1-20;
   p=1-3;
   L and Q are independently selected from the group consisting of C₁-C₆ alkyl, —X—(C═O)—Y—CH₂—, —X—(C═O)—, —X—CH₂—, where X and Y are independently selected from oxygen, NH and a direct bond;
   W is an amino, guanidino or amidino moiety;
   Z is a weakly basic moiety that has a pKₐ of less than 7.4 and greater than about 4.0; and
   (ii) a polyanionic compound.

In one embodiment, L and Q are C₁-C₆ alkyl. In another embodiment, p is 1 and W is —NR⁸₂—, wherein each R⁸ is independently selected from H or C₁₋₆ alkyl. In another embodiment, p is 2 and W is —NR⁸—.

In certain embodiments, n=1-10 or 1-5. In other embodiments, m=1-10 or 1-5.

In particular embodiments, the pKₐ of Z is less than 6.5 and greater than about 5.0. In certain other embodiments, the pKₐ of Z is less than 6.0 and greater than about 5.0. In certain embodiments, Z is a cyclic or acyclic amine, and in particular Z is imidazole.

In one embodiment, the polyanionic compound is a polynucleotide, a negatively charged protein, or a polysaccharide. In particular embodiments, the polynucleotide is a plasmid, DNA, RNA, a DNA/RNA hybrid, an oligonucleotide, an antisense oligonucleotide, a small interfering RNA, a polynucleotide analog having surrogate linkers, a hybrid polynucleotide comprising pentavalent phosphate linkers and surrogate linkers, or mixtures thereof. The polynucleotide can also comprise a modified nucleotide, a non-naturally occurring nucleotide, a protein-nucleic acid complex, or a polynucleotide-drug conjugate. Preferably, the polynucleotide is entrapped in at least a portion of the liposomes.

In additional embodiments, the composition further includes a therapeutic agent entrapped in the liposomes.

The liposomes can also include a lipopolymer (e.g., a lipid derivatized with a hydrophilic polymer) to form a surface coating of hydrophilic polymer chains. In particular, the lipopolymer comprises a hydrophilic polymer such as polyethyleneglycol, polyvinylpyrrolidone, polyvinylmethylether, polyhydroxypropyl methacrylate, polyhydroxyethyl methacrylate, polyhydroxyethyl acrylate, polymethacrylamide, polydimethylacrylamide, polymethyloxazoline, polyethyloxazoline, polyhydroxyproploxazoline, polyaspartamide, and polyethyleneoxide-polypropylene oxide, copolymers thereof and mixtures thereof. The hydrophilic polymer is covalently bound to the lipid, and in some embodiments, the covalent linkage is cleavable to allow detachment of the polymer from the liposome. Cleavage can be effected by acid, base, thiol, enzymatic action (e.g., a protease, esterase or glycosidase), oxidation, reduction, or light. Cleavable linkages include, without limitation, esters, hydrazones, disulfides, amides, and ethers.

In additional embodiments, the liposomes further comprise a ligand for targeting the liposomes to a target site. The targeting ligand can be attached directly to the polar headgroup of a liposome forming lipid, directly or via linkages known in the art. The targeting ligand can also be covalently attached to a distal end of the hydrophilic polymer on the lipopolymer. In particular, the targeting ligand has a binding affinity for the intended target cells, for example, endothelial cells, tumor cells, or cells for which gene therapy is desired, for internalization by such cells. The target cells are not limited to those enumerated herein, and one skilled in the art can select a target cell as desired for an intended treatment. In certain embodiments, the targeting ligand is a peptide, a saccharide, a vitamin (e.g., folate, biotin, cyanocobalamin), an antibody, a lectin, or mimetics thereof. In other embodiments, the targeting ligand specifically binds to an extracellular domain of a growth factor receptor. Such receptors are selected from c-erbB-2 protein product of the HER2/neu oncogene, epidermal growth factor receptor, basic fibroblast growth factor receptor, and vascular endothelial growth factor receptor. In another embodiment, the targeting ligand binds to a receptor selected from E-selectin receptor, L-selectin receptor, P-selectin receptor, folate receptor, CD4 receptor, CD19 receptor, a integrin receptors and chemokine receptors. The targeting ligand can also be, for example, folic acid, pyridoxal phosphate, vitamin B 12, sialyl Lewis^{x}, transferrin, epidermal growth factor, basic fibroblast growth factor, vascular endothelial growth factor, VCAM-1, ICAM-1, PECAM-1, an RGD peptide or an NGR peptide.

In certain embodiments, the liposomes include between 5-80 mole percent of the lipid of formula I. In other embodiments, the vesicle forming lipids comprise between 1-30 mole percent of a lipopolymer comprising a hydrophilic polymer, such as those listed above. The addition of the lipopolymer is effective to extend the circulation time of the liposomes when compared to liposomes lacking the lipopolymer. In yet other embodiments, the liposomes also include a cationic lipid.

In another aspect, a method is provided for preparing liposomes for administration of a polyanionic compound, where the liposomes are characterized by an extended blood circulation time. The method comprises forming liposomes from vesicle-forming lipids comprising a neutral cationic lipid having a structure according to formula (I) above, and adding a polyanionic compound. The liposomes are sized to a selected size in a range of between about 0.05 to 0.5 microns. The neutral cationic lipid is effective to extend the circulation time of the liposomes when compared to liposomes lacking the neutral cationic lipid.

In yet another aspect, a method is provided for transfecting a cell, comprising contacting a cell with the liposome compositions described herein. In another aspect, a method for delivering a polyanionic compound to a cell is provided, where a cell is contacted with the liposome compositions described herein.

### BRIEF DESCRIPTION OF THE DRAWINGS

FIG. 1 shows a synthetic scheme for preparation of distearoylphosphatidylethanolamine imidazole (DSPEI) and of distearoylphosphatidylethanolamine diimidazole (DSPEDI).

FIG. 2 shows zeta potential measurements as a function of pH for liposomes prepared from DSPEI, from a neutral cation lipid (NCL) containing histamine distearoyl glycerol (HDSG), and from dimethyldioctadecylammonium.

FIG. 3 shows the transfection of baby hamster kidney cells with DNA-liposome complexes.

### DETAILED DESCRIPTION OF THE INVENTION

I. Definitions

Before describing the present invention in detail, it is to be understood that unless otherwise indicated this invention is not limited to specific lipids or synthetic methods, as such may vary. It is also to be understood that the terminology used herein is for the purpose of describing particular embodiments only, and is not intended to be limiting. It must be noted that, as used in this specification and the appended claims, the singular forms “a,” “an,” and “the” include plural referents unless the context clearly dictates otherwise. Thus, for example, reference to “a polynucleotide” includes not only a single polynucleotide but also a combination or mixture of two or more different polynucleotide, and the like.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In describing and claiming the present invention, the following terminology will be used in accordance with the definitions set out below.

The definition of “cationic” refers to the property of having a net positive charge, and can include the presence of negative charges so long as the sum of charges present is positive.

The term “anionic” refers to the property of having a net negative charge, and similarly can include the presence of positive charges so long as the sum of charges present is negative.

The term “polyanionic” refers to compounds having the property of having more than one negative charge.

The term “polynucleotide” refers to a nucleic acid sequence that is at least 6 nucleotides in length, and includes DNA, RNA, RNA/DNA hybrids, catalytic RNA, nucleic acids containing non-naturally occurring nucleotides or modified nucleotides, oligonucleotides, antisense oligonucleotides, small interfering RNAs, triplex binding nucleic acid sequences, poly- or oligonucleotide analogs containing surrogate non-phosphodiester linkages, hybrid polynucleotides containing pentavalent phosphate linkers and surrogate linkages, such as peptide nucleic acid-nucleic acid hybrids, protein-nucleic acid complexes, or polynucleotide (or oligonucleotide)-drug conjugates and the like, so long as the polynucleotide retains a polyanionic character.

As used herein, a “neutral” lipid is one that has no net charge at neutral pH, and includes zwitterionic lipids, possessing equal numbers of positive and negative charges at neutral pH.

A “charged” lipid is one having a net positive or net negative charge.

A “lipopolymer” is a lipid derivatized with a hydrophilic polymer.

A “neutral cationic lipid” is generally a lipid that contains a weakly basic moiety that has no net charge in the pH range from about pH 7 to about 7.5, and becomes predominantly cationic at a pH below the pKₐ of the weakly basic moiety. Thus, the neutral cationic lipid is neutral at physiological pH, but is cationic at a pH less than the pKₐ of the basic group.

The term “liposome” is used in its conventional sense to refer to lipid vesicles, and also includes lipid-polynucleotide particles that might have a morphology different from a conventional lipid vesicle.

The term “vesicle-forming lipids” refers to amphipathic lipids which have hydrophobic and polar head group moieties, and which can form spontaneously into bilayer vesicles in water. Vesicle-forming lipids are exemplified by phospholipids, where when in the form of a bilayer vesicle, the hydrophobic moiety is in contact with the interior, hydrophobic region of the bilayer membrane, and the polar head group moiety is oriented toward the exterior, polar surface of the bilayer membrane. The vesicle-forming lipids of this type typically include one or two hydrophobic acyl hydrocarbon chains or a steroid group, and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation.

“Alkyl” refers to a fully saturated monovalent radical containing carbon and hydrogen, and which may be branched or a straight chain. Examples of alkyl groups are methyl, ethyl, n-butyl, t-butyl, n-heptyl, and isopropyl. “Lower alkyl” refers to an alkyl radical of one to six carbon atoms, as exemplified by methyl, ethyl, n-butyl, i-butyl, t-butyl, isoamyl, n-pentyl, and isopentyl.

“Alkenyl” refers to monovalent radical containing carbon and hydrogen, which may be branched or a straight chain, and which contains one or more double bonds.

“Hydrophilic polymer” as used herein refers to a polymer having moieties soluble in water, which lend to the polymer some degree of water solubility at room temperature. Exemplary hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, polyethyleneoxide-polypropylene oxide copolymers, copolymers of the above-recited polymers, and mixtures thereof. Properties and reactions with many of these polymers are described in U.S. Pat. Nos. 5,395,619 and 5,631,018.

A “functionalized polymer” is a polymer containing one or more reactive functional groups and refers to a polymer that has been modified, typically but not necessarily, at a terminal end moiety for reaction with another compound to form a covalent linkage. Reaction schemes to functionalize a polymer to have such a reactive functional group of moiety are readily determined by those of skill in the art and/or have been described, for example in U.S. Pat. No. 5,613,018 or by Zalipsky et al., in for example, Eur. Polymer. J., 19(12):1177-1183 (1983); Bioconj. Chem., 4(4):296-299 (1993).

Abbreviations: PEG: polyethylene glycol; mPEG: methoxy-terminated polyethylene glycol; Chol: cholesterol; PC: phosphatidyl choline; PHPC: partially hydrogenated phosphatidyl choline; PHEPC: partially hydrogenated egg phosphatidyl choline; PHSPC: partially hydrogenated soy phosphatidyl choline; DSPE: distearoyl phosphatidyl ethanolamine; DSPEI: distearoyl phosphoethanolamine imidazole; APD: 1-amino-2,3-propanediol; DTPA: diethylenetetramine pentaacetic acid; Bn: benzyl; NCL: neutral cationic liposome; FGF: fibroblast growth factor; HDSG; histamine distearoyl glycerol; DOTAP: 1,2-diolelyloxy-3-(trimethylamino)propane; DTB: dithiobenzyl; FC-PEG: fast-cleavable PEG; SC-PEG: slow-cleavable PEG; DDAB: dimethyldioctadecylammonium; EtDTB, ethyl-dithiobenzyl; DOPE, dioleoyl phosphatidylethanolamine; BHK, baby hamster kidney.

II. Liposomes

In one aspect, the invention includes a liposome composition comprised of liposomes and a polyanionic compound, preferably a polynucleotide. The liposomes comprise a neutral cationic lipid, and optionally a lipopolymer, optionally derivatized through a releasable bond. The liposome can also comprise a targeting ligand. These liposome components will now be described.

A. Neutral Cationic Lipid

The neutral cationic lipid included in the liposomes of the present invention is generally a lipid represented by a structure according to formula (I): 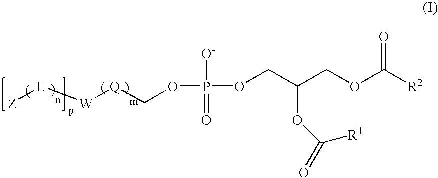
wherein each of R¹ and R² is a branched or unbranched alkyl, alkenyl or alkynyl chain having between 6-24 carbon atoms;
n=1-20;
m=1-20;
p=1-3;
L and Q are independently selected from the group consisting of C₁-C₆ alkyl, —X—(C═O)—Y—CH₂—, —X—(C═O)—, —X—CH₂—, where X and Y are independently selected from oxygen, NH and a direct bond;
W is an amino, guanidino or amidino moiety; and
Z is a weakly basic moiety that has a pKₐ of less than 7.4 and greater than about 4.0.

In another embodiment, Z is a moiety having a pKₐ value between 4.5-7.0, more preferably between 5-6.5, and most preferably between 5-6.

The weakly basic moiety Z results in a lipid that at physiologic pH of 7.4 is predominantly, e.g., greater than 50%, neutral in charge but at a selected pH value lower than its pKₐ, tends to have a predominantly positive charge. By way of example, and in a preferred embodiment, Z is an imidazole moiety, which has a pKa of about 6.0. At physiologic pH of 7.4, this moiety is predominantly neutral, but at pH values lower than 6.0, the moiety becomes predominantly positive. In support of the invention, a lipid having an imidazole moiety was prepared and used in preparation of liposomes, as will be discussed below.

In addition to imidazole, other cyclic amines such as substituted imidazoles, as well as benzimidazoles and naphthimidazoles, can be used as the Z moiety in the structure given above, as long as the substitution does not alter the pKa to a value outside the desired range. Suitable substituents typically include alkyl, hydroxyalkyl, alkoxy, aryl, halogen, haloalkyl, amino, and aminoalkyl. Examples of such compounds reported to have pKa's in the range of 5.0 to 6.0 include, but are not limited to, various methyl-substituted imidazoles and benzimidazoles, histamine, naphth[1,2-d]imidazole, 1H-naphth[2,3-d]imidazole, 2-phenylimidazole, 2-benzyl benzimidazole, 2,4-diphenyl-1H-imidazole, 4,5-diphenyl-1H-imidazole, 3-methyl-4(5)-chloro-1H-imidazole, 5(6)-fluoro-1H-benzimidazole, and 5-chloro-2-methyl-1H-benzimidazole.

Other nitrogen-containing cycliq amines such as heteroaromatics, including pyridines, quinolines, isoquinolines, pyrimidines, phenanthrolines, and pyrazoles, can also be used as the Z group. Again, many such compounds having substituents selected from alkyl, hydroxyalkyl, alkoxy, aryl, halo, alkyl, amino, aminoalkyl, and hydroxy are reported to have pK's in the desired range. These include, among pyridines, 2-benzylpyridine, various methyl- and dimethylpyridines, as well as other lower alkyl and hydroxylalkyl pyridines, 3-aminopyridine, 4-(4-aminophenyl)pyridine, 2-(2-methoxyethyl)pyridine, 2-(4-aminophenyl)pyridine, 2-amino-4-chloropyridine, 4-(3-furanyl)pyridine, 4-vinylpyridine, and 4,4′-diamino-2,2′-bipyridine, all of which have reported pKa's between 5.0 and 6.0. Quinolinoid compounds reported to have pKa's in the desired range include, but are not limited to, 3-, 4-, 5-, 6-, 7- and 8-amino isoquinoline, various lower alkyl- and hydroxy-substituted quinolines and isoquinolines, 4-, 5-, 7- and 8-isoquinolinol, 5-, 6-, 7- and 8-quinolinol, 8-hydrazinoquinoline, 2-amino-4-methylquinazoline, 1,2,3,4-tetrahydro-8-quinolinol, 1,3-isoquinolinediamine, 2,4-quinolinediol, 5-amino-8-hydroxyquinoline, and quinuclidine. Also having pKa's in the desired range are several amine-substituted pyrimidines, such as 4-(N,N-dimethylamino)pyrimidine, 4-(N-methylamino)pyrimidine, 4,5-pyrimidine diamine, 2-amino-4-methoxy pyrimidine, 2,4-diamino-5-chloropyrimidine, 4-amino-6-methylpyrimidine, 4-amino pyrimidine, and 4,6-pyrimidinediamine, as well as 4,6-pyrimidinediol. Various phenanthrolines, such as 1,10-, 1,8-, 1,9-, 2,8-, 2,9- and 3,7-phenanthroline, have pKa's in the desired range, as do most of their lower alkyl-, hydroxyl-, and aryl-substituted derivatives. Pyrazoles which may be used include, but are not limited to, 4,5-dihydro-1H-pyrazole, 4,5-dihydro-4-methyl-3H-pyrazole, 1-hydroxy-1H-pyrazole, and 4-aminopyrazole.

Many nitrogen-substituted aromatics, such as anilines and naphthylamines, are also suitable embodiments of the group Z. Anilines and naphthylamines further substituted with groups selected from methyl or other lower alkyl, hydroxyalkyl, alkoxy, hydroxyl, additional amine groups, aminoalkyl, halogen, and haloalkyl are generally reported to have pKa's in the desired range. Other amine-substituted aromatics which can be used include 2-aminophenazine, 2,3-pyrazinediamine, 4- and 5-aminoacenaphthene, 3- and 4-amino pyridazine, 2-amino-4-methylquinazoline, 5-aminoindane, 5-aminoindazole, 3,3′,4,4′-biphenyl tetramine, and 1,2- and 2,3-diaminoanthraquinone.

Also included as embodiments of Z are certain acyclic amine compounds, such as various substituted hydrazines, including trimethylhydrazine, tetramethylhydrazine, 1-methyl-1-phenylhydrazine, 1-naphthalenylhydrazine, and 2-, 3-, and 4-methylphenyl hydrazine, all of which are reported to have pKa's between 4.5 and 7.0. Alicyclic compounds having pKa's in this range include 1-pyrrolidineethanamine, 1-piperidineethanamine, hexamethylenetetramine, and 1,5-diazabicyclo[3.3.3]undecane.

Also suitable as the Z moiety in the structure given above are certain aminosugars, as described in copending U.S. Patent Application Publication No. U.S. 2003/0031764.

The above listings give examples of compounds having pKa's between 4.5 and 7.0 which may be used as pH-responsive groups in the lipid conjugates of the invention; these listings are not intended to be limiting. In selected embodiments, the group Z is a imidazole, aniline, aminosugar or derivative thereof. Preferably, the effective pKa of the group Z is not significantly affected by its attachment to the lipid group. Examples of linked conjugates are given below.

The lipids of the invention include a neutral linkage L joining the Z moiety and the quaternary ammonium moiety, W. The lipids also include a neutral linkage Q between the quaternary ammonium moiety, W, and the phosphate moiety of the phospholipid head group. Linkages L and Q are variable, and in preferred embodiments each is selected from a methylene, a carbamate, an ester, an amide, a carbonate, a urea, an amine, and an ether. In a preferred lipid prepared in support of the invention, methylene linkages, where L and Q are —CH₂—, was prepared.

In the tail portion of the lipid, R¹ and R² are the same or different and can be a branched or an unbranched alkyl, alkenyl, or alkynyl chain having between 6-24 carbon atoms. More preferably, the R¹ and R² groups are between 12-22 carbon atoms in length, with R¹═R²═C₁₇H₃₅ (such that the group is a stearyl group) or R¹═R²═C₁₇H₃₃ (such that the group is an oleoyl group), or R¹═R²═C₁₅H₃₃ (comprising palmitoyl chains).

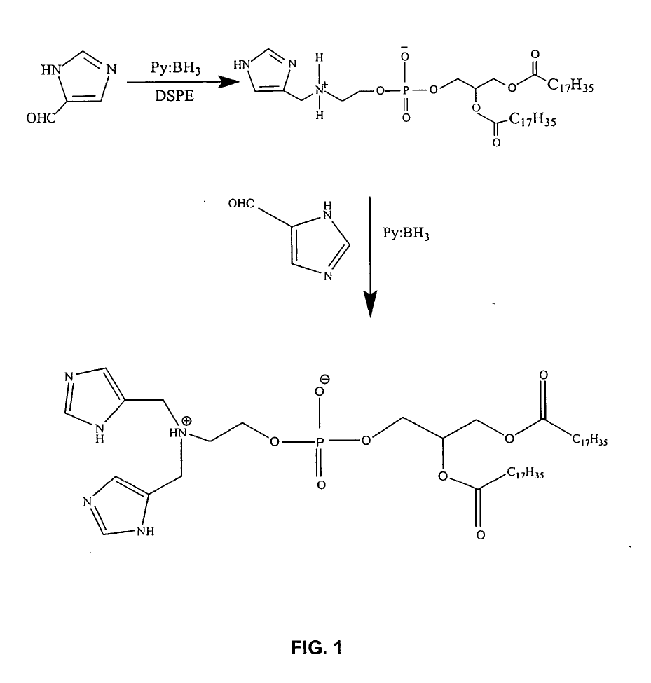

The lipids of the invention can be prepared using standard synthetic methods. As mentioned above, in studies performed in support of the invention, a lipid having the structure shown above, where Z is an imidazole, n=1, p=1, m=1, L is a methylene, Q is a methylene, W is amino, and R¹═R²═C₁₇H₃₅, was prepared. A reaction scheme for preparation of the exemplary lipid is illustrated in FIG. 1 and details of the synthesis are provided in Example 1. Briefly, the distearoylphosphatidylethanolamine imidazole was prepared from distearoylphosphatidylethanolamine and 4(5)-imidazole carboxaldehyde and reacted in the presence of pyridine/borane to yield a lipid having an imidazole moiety linked to the amino moiety of phosphatidylethanolamine via a methylene linkage. When an excess of aldehyde is used, two imidazoles become linked to phosphatidylethanolamine, yielding the diimidazole. A similar route, using a benzimidazole carboxaldehyde in place of 4(5)-imidazole carboxaldehyde, can be used to produce a benzimidazole linked phosphatidylethanolamine.

Preparation of the lipid having other linkages is readily done by those of skill in the art using conventional methods. Other linkages include ether (L=—O—CH₂—) and ester linkages (L=—O—(C═O)—), as well as amide, urea and amine linkages (i.e., where L=—NH—(C═O)—NH—, —NH—(C═O)—CH₂—, —NH—(C═O)—NH—CH₂—, or —NH—CH₂—). Additional details of synthetic procedures can be obtained using conventional methods, and for example, from co-pending co-owned U.S. Patent Application Publication No. U.S. 2003/0031764.

In a study conducted in support of the invention, liposomes comprised of DSPEI were prepared as described in Example 3. For comparison, liposomes comprised of a neutral cationic lipid described in copending U.S. Patent Application Publication No. U.S. 2003/0031764, histamine-distearoyl glycerol (HDSG) were also prepared. The imidazole of histamine has a pKa of 6. HDSG tends to neutral at physiological pH (pH 7.4), and is predominantly positively charged at a pH lower than 6. Liposomes composed of HDSG encapsulate DNA at about pH 4 to 5, similar to conventional cationic liposomes. The surface charge of the HDSG liposome/complex is reduced at physiological pH in the blood circulation. The surface charge of HDSG is predominantly positive at pH 5 to 6 (the consensus pH in endosome and lysosome) to facilitate the interaction of the complexes with the lysosomal membrane and release of the nucleic acid content into the cytoplasm.

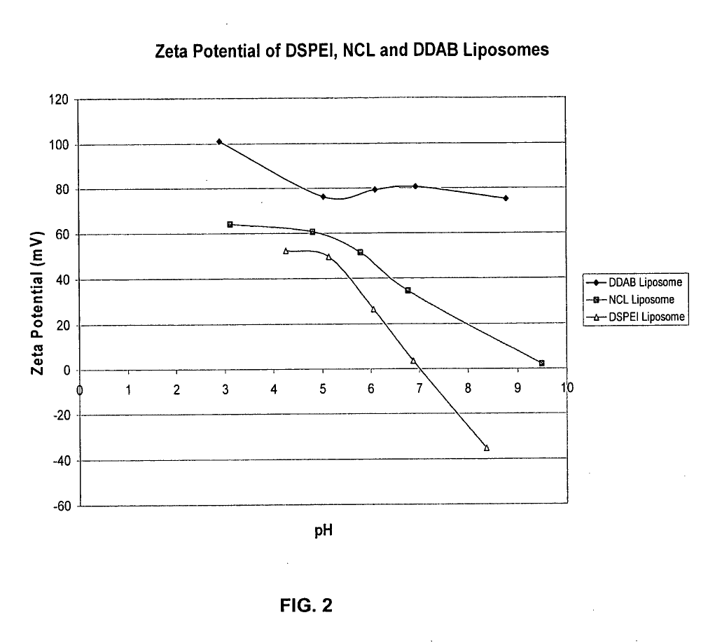

As discussed in Example 5, zeta potential measurements were obtained for the liposomes containing DSPEI and for the liposomes containing HDSG. The results are shown in FIG. 2. The zeta potential for DSPEI-containing liposomes (triangles) is zero near physiological pH, indicating that the DSPEI-containing liposomes were neutral near pH 7. The decrease in zeta potential with increasing pH for the DSPEI-containing liposomes is much greater than observed for the other liposome preparations. The zeta potential for HDSG-containing liposomes (squares) was less responsive to changes in pH, as evidenced by a shallow zeta potential vs. pH slope. This is likely indicative of a higher pKa and greater charge at physiological pH. These results indicate that DSPEI-containing liposomes have a lower pKa and are more neutral at physiological pH than liposomes containing the neutral cationic lipid histamine distearoylglycerol (HDSG). The steeper slope for zeta potential versus pH for DSPEI relative to HDSG also indicates that DSPEI has a lower pKa than HDSG, and thus DSPEI-containing liposomes are even more neutral at physiological pH than HDSG-containing liposomes. The greater neutrality of DSPEI-containing liposomes is important for minimization of non-specific interactions with plasma proteins and cells under in vivo conditions and thus prolonged circulation in the blood, which is necessary for systemic drug and gene delivery, as well as delivery of gene modulators, to diseased tissues.

With continuing reference to FIG. 2, zeta potential measurements were also determined for liposomes prepared using dimethyldioctadecylammonium bromide (DDAB) (diamonds). The relatively flat slope of DDAB-containing liposomes indicates that there is little change in zeta potential with varying pH, and that the pKa for DDAB is higher than for either HDSG or DSPEI. Therefore DDAB-containing liposomes retain their cationic charge at physiological pH and are more likely to participate in non-specific interactions with plasma proteins under in vivo conditions. DDAB-containing liposomes are consequently cleared rapidly from circulation and are less suitable for drug or gene delivery to diseased tissues.

Additional advantages conferred by the neutral cationic lipids of formula (1) relate to the greater solubility of these lipids due to the presence of a polar head group. Greater solubility permits liposome DNA formulation at pH values closer to physiological pH. Also, lipids with a polar head group tend to pack better into lipid bilayers comprised of conventional phospholipids. The better packing imparts liposome stability.

While not wishing to be bound by theory, it is hypothesized that the neutral cationic lipids of formula (I) provide liposomes having increased stability on administration in vivo, and further provide uncharged liposomes at physiological pH that remain effective to entrap and deliver polyanionic compounds, yet evade non-specific interactions (e.g., with plasma proteins), and thus provide prolonged circulation in plasma. Thus, the neutral cationic lipids described herein are an improvement over the prior art cationic lipids and their associated risks of toxicity.

B. Vesicle-Forming Lipids

Vesicle-forming lipids are preferably ones having two hydrocarbon chains, typically acyl chains, and a polar head group. Included in this class are the phospholipids, such as phosphatidylcholine (PC), phosphatidic acid (PA), phosphatidylinositol (PI), and sphingomyelin (SM), where the two hydrocarbon chains are typically between about 14-22 carbon atoms in length, and have varying degrees of unsaturation. In some instances, it may be desirable to include vesicle-forming lipids having branched hydrocarbon chains.

The above-described lipids and phospholipids whose acyl chains have a variety of degrees of saturation can be obtained commercially, or prepared according to published methods. Other lipids that can be included in the invention are glycolipids and sterols, such as cholesterol. Commercially available products, such as egg or soy phosphatidylcholine, can be utilized in a partially hydrogenated state or a natural state. In the examples below, partially hydrogenated soy phosphatidylcholine was utilized (PHSPC).

The different types of vesicle forming lipids can also be mixed, so that for example, liposomes can be prepared using a wide variety of lipids, present in various mole fractions. For example, liposomes are commonly prepared from mixtures of PE, PC and cholesterol.

C. Lipopolymers: Lipid Derivatized with a Hydrophilic Polymer

A second component which can optionally be included in the liposome composition is a lipopolymer, or lipid derivatized with a hydrophilic polymer. The vesicle-forming lipids which can be used as lipopolymers are any of those described for the vesicle-forming lipid component. Vesicle forming lipids with diacyl chains, such as phospholipids, are preferred. One exemplary phospholipid is phosphatidylethanolamine (PE), which provides a reactive amino group which is convenient for coupling to the activated polymers. An exemplary PE is distearyl PE (DSPE). Derivatization with polyethyleneglycol yields a preferred lipopolymer, methoxy-PEG-DSPE, preferably derivatized via a urethane linkage.

The incorporation of lipopolymer into a liposome can present significant advantages, such as reduced leakage of an encapsulated drug. Additionally, another advantage is a greater flexibility in modulating interactions of the liposomal surface with target cells and with the RES (Miller et al., Biochemistry, 37:12875-12883 (1998)). PEG-substituted synthetic ceramides have been used as uncharged components of sterically stabilized liposomes (Webb et al., Biochim. Biophys. Acta, 1372:272-282 (1998)); however, these molecules are complex and expensive to prepare, and they generally do not pack into the phospholipid bilayer as well as diacyl glycerophospholipids.

Lipopolymers as described in U.S. Pat. No. 6,586,001 to Zalipsky can also be utilized, and present certain advantages over the PEG-substituted synthetic ceramides in ease of preparation and cost. The lipopolymers described in U.S. Pat. No. 6,586,001 include a neutral linkage in place of the charged phosphate linkage of PEG-phospholipids, such as PEG-DSPE, which are frequently employed in sterically stabilized liposomes. This neutral linkage is typically selected from a carbamate, an ester, an amide, a carbonate, a urea, an amine, and an ether. Hydrolyzable or otherwise cleavable linkages, such as disulfides, hydrazones, peptides, carbonates, and esters, are preferred in applications where it is desirable to remove the PEG chains after a given circulation time in vivo. This feature can be useful in releasing drug or facilitating uptake into cells after the liposome has reached its target (Martin et al., U.S. Pat. No. 5,891,468; Zalipsky et al., PCT Publication No. WO 98/18813 (1998)) or in temporarily masking a targeting ligand.

Exemplary hydrophilic polymers include polyethyleneglycol, polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, polyethyleneoxide-polypropylene oxide copolymers, copolymers of the above-recited polymers, and mixtures thereof. Properties and reactions with many of these polymers are described in U.S. Pat. Nos. 5,395,619 and 5,631,018. Other polymers which may be suitable include polylactic acid, polyglycolic acid, and copolymers thereof, as well as derivatized celluloses, such as hydroxymethylcellulose or hydroxyethylcellulose. Additionally, block copolymers or random copolymers of these polymers, particularly including PEG segments, may be suitable. Methods for preparing lipids derivatized with hydrophilic polymers, such as PEG, are well known e.g., as described in co-owned U.S. Pat. No. 5,013,556.

The preferred polymer in the derivatized lipid, is polyethyleneglycol (PEG), preferably a PEG chain having a molecular weight between 1,000-15,000 daltons, more preferably between 1,000 and 5,000 daltons.

In particular embodiments, the hydrophilic polymer is attached via a releasable bond, such as a dithiobenzyl moiety, described in U.S. Patent Application Publication No. U.S. 2003/0031764 and in U.S. Pat. No. 6,342,244 to Zalipsky.

As will be described below, liposomes comprised of the neutral cationic lipid were prepared in studies in support of the invention. Lipopolymers were included in certain examples.

D. Targeting Ligands

The liposomes may optionally be prepared to contain surface groups, such as antibodies or antibody fragments, small effector molecules for interacting with cell-surface receptors, antigens, and other like compounds, for achieving desired target-binding properties to specific cell populations. Such ligands can be included in the liposomes by including in the liposomal lipids a lipid derivatized with the targeting molecule, or a lipid having a polar headgroup that can be derivatized with the targeting molecule in preformed liposomes (e.g., phosphatidylethanolamine having a reactive amino moiety). Alternatively, a targeting moiety can be inserted into preformed liposomes by incubating the preformed liposomes with a ligand-polymer-lipid conjugate.

Lipids can be derivatized with the targeting ligand by covalently attaching the ligand to the free distal end of a hydrophilic polymer chain, which is attached at its proximal end to a vesicle-forming lipid, and incorporating the targeting ligand into liposomes (Zalipsky, S., (1997) Bioconjugate Chem., 8(2):111-118). Alternatively, the targeting ligand can be derivatized to a lipid (e.g., phosphatidylethanolamine) directly or through a linking group, thereby remaining masked until removal of the hydrophilic polymer chains. Of course, it will be appreciated by one skilled in the art that it may be desired at times to incorporate the targeting ligand into the liposome without the presence of the lipopolymer.

There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Zalipsky, S., (1997) Bioconjugate Chem., 8(2):111-118; Allen, T. M., et al., (1995) Biochemicia et Biophysica Acta 1237:99-108; Zalipsky, S., (1993) Bioconjugate Chem., 4(4):296-299; Zalipsky, S., et al., (1994) FEBS Lett. 353:71-74; Zalipsky, S., et al., (1995) Bioconjugate Chemistry, 705-708; Zalipsky, S., in STEALTH LIPOSOMES (D. Lasic and F. Martin, Eds.) Chapter 9, CRC Press, Boca Raton, Fla. (1995)).

Targeting ligands are well known to those of skill in the art, and in a preferred embodiment of the present invention, the ligand is one that has binding affinity to endothelial or tumor cells, and which can be, in one embodiment, internalized by the cells. Such ligands often bind to an extracellular domain of a growth factor receptor. Targeting ligands include, without limitation, peptides, saccharides, vitamins, antibodies or antibody fragments, lectins, receptor ligands, or mimetics thereof. In particular embodiments, the targeting ligand specifically binds to an extracellular domain of a growth factor receptor. Such receptors are selected from c-erbB-2 protein product of the HER2/neu oncogene, epidermal growth factor receptor, basic fibroblast growth factor receptor, and vascular endothelial growth factor receptor. In another embodiment, the targeting ligand binds to a receptor selected from E-selectin receptor, L-selectin receptor, P-selectin receptor, folate receptor, CD4 receptor, CD19 receptor, αβ integrin receptors and chemokine receptors. The targeting ligand can also be folic acid, biotin, pyridoxal phosphate, vitamin B12 (cyanocobalamin), sialyl Lewis^{x}, transferrin, epidermal growth factor, basic fibroblast growth factor, vascular endothelial growth factor, VCAM-1, ICAM-1, PECAM-1, an RGD peptide or an NGR peptide. In certain other embodiments, the ligand is E-selectin, Her-2 or FGF.

III. Polyanionic Compounds

Polyanionic compounds that can be included in the compositions described herein include polynucleotides, polynucleotide analogs having surrogate linkers, negatively charged proteins, or polysaccharides.

A. Polynucleotides and Polynucleotide Analogs

The polynucleotide can be a plasmid, DNA, RNA, a DNA/RNA hybrid, an oligonucleotide, an antisense oligonucleotide, a small interfering RNA, or a hybrid polynucleotide comprising pentavalent phosphate linkers as well as surrogate linkers. The polynucleotide can also comprise a modified nucleotide, a non-naturally occurring nucleotide, a protein-nucleic acid complex, or a polynucleotide-drug conjugate. Preferably, the polynucleotide is entrapped in at least a portion of the liposomes.

As used herein, the terms “nucleoside” and “nucleotide” refer to nucleosides and nucleotides containing not only the conventional purine and pyrimidine bases, i.e., adenine (A), thymine (T), cytosine (C), guanine (G), and uracil (U), but also modified nucleosides and nucleotides. Such modifications include, but are not limited to, methylation or acylation of a purine or pyrimidine moiety, substitution of a different heterocyclic ring structure for a pyrimidine ring or for one or both rings in the purine ring system, and protection of one or more functionalities, e.g., using a protecting group such as acetyl, difluoroacetyl, trifluoroacetyl, isobutyryl, benzoyl, and the like. Modified nucleosides and nucleotides also include modifications on the sugar moiety, e.g., wherein one or more of the hydroxyl groups are replaced with halide and/or hydrocarbyl substituents (typically aliphatic groups, in the latter case), or are functionalized as ethers, amines, or the like. Common analogs include, but are not limited to, 1-methyladenine, 2-methyladenine, N⁶-methyladenine, N⁶-isopentyl-adenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methyl-aminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine, and 2,6-diaminopurine. Iso-guanine and iso-cytosine may be incorporated into oligonucleotides to lower potential cross reactivity between sequences when hybridization is not desired.

As used herein, the term “polynucleotide” also encompasses polydeoxyribonucleotides (containing 2-deoxy-D-ribose), polyribonucleotides (containing D-ribose), any other type of polynucleotide analog having surrogate linkers, such as N-glycoside of a purine or pyrimidine base, and other polymers containing nonnucleotidic backbones (e.g., phosphorothioates, phosphorodithioates, peptide nucleic acids and synthetic sequence-specific nucleic acid polymers commercially available from the Anti-Gene Development Group, Corvallis, Oreg., as Neugene™ polymers) or other surrogate linkages, providing that the polymers contain nucleobases in a configuration that allows for base pairing and base stacking, such as is found in DNA and RNA. Thus, “oligonucleotides” herein include double- and single-stranded DNA, as well as double- and single-stranded RNA and DNA/RNA hybrids, and also include known types of modified oligonucleotides, such as, for example, oligonucleotides wherein one or more of the naturally occurring nucleotides is substituted with an analog; oligonucleotides containing surrogate linkages such as, for example, those with uncharged linkages (e.g., methyl phosphonates, phosphotriesters, phosphoramidates, carbamates, etc.), negatively charged linkages (e.g., phosphorothioates, phosphorodithioates, phosphoroselenoates, etc.), and positively charged linkages (e.g., aminoalkylphosphoramidates, aminoalkylphosphotriesters), those containing pendant moieties, such as, for example, proteins (including nucleases, toxins, antibodies, peptides), intercalators (e.g., acridine, psoralen, etc.), chelators (e.g., metals, radioactive metals, boron, oxidative metals, etc.), alkylating agents, dyes or fluorescent labels, or oligonucleotide-drug conjugates, as described in Byrn, S. R., et al., (1991) in “Drug-oligonucleotide conjugates,” Adv. Drug Delivery Reviews 6: 287-308.

There is no intended distinction in length between the terms “polynucleotide” and “oligonucleotide,” and these terms are used interchangeably. As used herein the symbols for nucleotides and polynucleotides are according to the IUPAC-IUB Commission of Biochemical Nomenclature recommendations (Biochemistry 9:4022, 1970).

Oligonucleotides can be synthesized by known methods. Background references that relate generally to methods for synthesizing oligonucleotides include those related to 5′-to-3′ syntheses based on the use of β-cyanoethyl phosphate protecting groups, e.g., de Napoli et al. (1984) Gazz. Chim. Ital. 114:65, Rosenthal et al. (1983) Tetrahedron Lett. 24:1691, Belagaje and Brush (1977) Nuc. Acids Res. 10:6295, in references which describe solution-phase 5′-to-3′ syntheses include Hayatsu and Khorana (1957) J. Am. Chem. Soc. 89:3880, Gait and Sheppard (1977) Nuc. Acids Res. 4: 1135, Cramer and Koster (1968) Angew. Chem. Int. Ed. Engl. 7:473, and Blackburn et al. (1967), J. Chem. Soc. Part C, at 2438. Additionally, Matteucci and Caruthers (1981) J. Am. Chem. Soc. 103:3185-91 described the use of phosphochloridites in the preparation of oligonucleotides. Beaucage and Caruthers (1981) Tetrahedron Lett. 22:1859-62, and U.S. Pat. No. 4,415,732 described the use of phosphoramidites for the preparation of oligonucleotides. Smith, ABL 15-24 (December 1983) describes automated solid-phase oligodeoxyribonucleotide synthesis. See also the references cited therein, and Warner et al. (1984) DNA 3:401-11. T. Horn and M. S. Urdea (1986) DNA 5:421-25 described phosphorylation of solid-supported DNA fragments using bis(cyanoethoxy)-N,N-diisopropylaminophosphine. See also, T. Horn and M. S. Urdea (1986) Tetrahedron Lett. 27:4705-08.

The liposomes formed of the lipids described above are associated with a nucleic acid. By “associated” it is meant that a therapeutic agent, such as a nucleic acid, is entrapped in the liposomes central compartment and/or lipid bilayer spaces, is associated with the external liposome surface, or is both entrapped internally and externally associated with the liposomes. It will be appreciated that the therapeutic agent can be a nucleic acid or a drug compound. It will also be appreciated that a drug compound can be entrapped in the liposomes and a nucleic acid externally associated with the liposomes, or vice versa. The terms entrapped and associated are used interchangeably herein.

The nucleic acid can be selected from a variety of DNA and RNA based nucleic acids, including fragments and analogues of these. A variety of genes for treatment of various conditions have been described, and coding sequences for specific genes of interest can be retrieved from DNA sequence databanks, such as GenBank or EMBL. For example, polynucleotides for treatment of viral, malignant and inflammatory diseases and conditions, such as, cystic fibrosis, adenosine deaminase deficiency and AIDS, have been described. Treatment of cancers by administration of tumor suppressor genes, such as APC, DPC4, NF-1, NF-2, MTS1, RB, p53, WT1, BRCA1, BRCA2 and VHL, are contemplated.

Examples of specific nucleic acids for treatment of an indicated conditions include: HLA-B7, tumors, colorectal carcinoma, melanoma; IL-2, cancers, especially breast cancer, lung cancer, and tumors; IL-4, cancer; TNF, cancer; IGF-1 antisense, brain tumors; IFN, neuroblastoma; GM-CSF, renal cell carcinoma; MDR-1, cancer, especially advanced cancer, breast and ovarian cancers; and HSV thymidine kinase, brain tumors, head and neck tumors, mesothelioma, ovarian cancer.

The polynucleotide can be an antisense DNA oligonucleotide composed of sequences complementary to its target, usually a messenger RNA (mRNA) or an mRNA precursor. The mRNA contains genetic information in the functional, or sense, orientation and binding of the antisense oligonucleotide inactivates the intended mRNA and prevents its translation into protein. Such antisense molecules are determined based on biochemical experiments showing that proteins are translated from specific RNAs and once the sequence of the RNA is known, an antisense molecule that will bind to it through complementary Watson-Crick base pairs can be designed. Such antisense molecules typically contain between 10-30 base pairs, more preferably between 10-25, and most preferably between 15-20. The antisense oligonucleotide can be modified for improved resistance to nuclease hydrolysis, and such analogues include phosphorothioate, methylphosphonate, phosphoroselenoate, phosphodiester and p-ethoxy oligonucleotides (WO 97/07784).

The entrapped agent can also be a ribozyme, DNAzyme, catalytic RNA, or a small interfering RNA (siRNA) which induces RNA interference. RNA interference refers to the potent and specific gene silencing induced through a process referred to as RNA interference (RNAi) mediated through double-stranded RNA. RNAi is mediated by the RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. RNAi has become the method of choice for loss-of-function investigations in numerous systems including, C. elegans, Drosophila, fungi, plants, and even mammalian cell lines. To specifically silence a gene in most mammalian cell lines, small interfering RNAs (siRNA) are used because large dsRNAs (>30 bp) trigger the interferon response and cause nonspecific gene silencing.

Further background on RNA interference can be obtained from a review of the relevant literature: WO 01/68836; Bernstein et al., RNA (2001) 7: 1509-1521; Bernstein et al., Nature (2001) 409:363-366; Billy et al., Proc. Nat'l Acad. Sci USA (2001) 98:14428-33; Caplan et al., Proc. Nat'l Acad. Sci USA (2001) 98:9742-7; Carthew et al., Curr. Opin. Cell Biol (2001) 13: 244-8; Elbashir et al., Nature (2001) 411: 494-498; Hammond et al., Science (2001) 293:1146-50; Hammond et al., Nat. Ref. Genet. (2001) 2:110-119; Hammond et al., Nature (2000) 404:293-296; McCaffrrey et al., Nature (2002): 418-38-39; and McCaffrey et al., Mol. Ther. (2002) 5:676-684; Paddison et al., Genes Dev. (2002) 16:948-958; Paddison et al., Proc. Nat'l Acad. Sci USA (2002) 99:1443-48; Sui et al., Proc. Nat'l Acad. Sci USA (2002) 99:5515-20.

U.S. patents of interest in the field of RNA interference include U.S. Pat. Nos. 5,985,847 and 5,922,687. Also of interest is WO/I 1092. Additional references of interest include: Acsadi et al., New Biol. (January 1991) 3:71-81; Chang et al., J. Virol. (2001) 75:3469-3473; Hickman et al., Hum. Gen. Ther. (1994) 5:1477-1483; Liu et al., Gene Ther. (1999) 6:1258-1266; Wolff et al., Science (1990) 247: 1465-1468; and Zhang et al., Hum. Gene Ther. (1999) 10: 1735-1737: and Zhang et al., Gene Ther. (1999) 7:1344-1349.

The polyanionic compound preferably is a polynucleotide, and includes but is not limited to a plasmid (encoding, e.g., a gene), DNA, RNA, a DNA/RNA hybrid, an oligonucleotide, an antisense oligonucleotide, a small interfering RNA, a modified nucleotide, a non-naturally occurring nucleotide, or a protein-nucleic acid complex.

In one embodiment, the polynucleotide can be inserted into a plasmid, preferably one that is a circularized or closed double-stranded molecule having sizes preferably in the 5-40 Kbp (kilo basepair) range. Such plasmids are constructed according to well-known methods and include a therapeutic gene, i.e., the gene to be expressed in gene therapy, under the control of suitable promoter and enhancer, and other elements necessary for replication within the host cell and/or integration into the host-cell genome. Methods for preparing plasmids useful for gene therapy are widely known and referenced.

Polynucleotides, oligonucleotides, and other nucleic acids, as discussed above, can be entrapped in the liposome by passive entrapment during hydration of the lipid film. Other procedures for entrapping polynucleotides include condensing the nucleic acid in single-molecule form, where the nucleic acid is suspended in an aqueous medium containing protamine sulfate, spermine, spermidine, histone, lysine, cationic peptides, mixtures thereof, or other suitable polycationic condensing agent, under conditions effective to condense the nucleic acid into small particles. The solution of condensed nucleic acid molecules is used to rehydrate a dried lipid film to form liposomes with the condensed nucleic acid in entrapped form.

B. Negatively Charged Proteins

Negatively charged proteins include anionic proteins in the most general sense, so long as the protein is capable of interacting with the liposome comprising a neutral cationic lipid. The negatively charged proteins can be of any length, within the practical constraints of solubility. A preferred embodiment is a drug-protein conjugate, wherein the negatively charged protein provides a means for interacting with the liposome comprising a neutral cationic lipid. Negatively charged proteins include, without limitation, peptides in the polyglutamate or polyaspartate family, that is, containing one or more sequence motifs that are predominantly glutamate or aspartate residues; collagen, and albumin. Polyglutamic acid and polyaspartic acid drug carriers or conjugates, have been described by Li, C., (2002) Adv. Drug Delivery Reviews 54, 695-713 and Peterson, R. V., in “Biodegradable Drug Delivery Systems Based on Polypeptides,” in Bioactive Polymeric Systems: An Overview, Gerberin, C. G. & Carraher, C. R., Eds., Plenum Press, NY (1985). For example, polyglutamic acid conjugates of doxorubicin, daunorubicin, ara-C, uracil and uridine derivatives, cyclophosphamide, melphalan, mitomycin C, paclitaxel, and camptothecin can be prepared and delivered using liposomes comprising the neutral cationic lipid described herein.

C. Polysaccharides

Negatively charged polysaccharides are also included within the polyanionic compounds that can be used in the present composition with liposomes comprising a neutral cationic lipid. Sulfated polysaccharides are an exemplary class of negatively charged polysaccharides, and include, without limitation, heparin sulfate, hyaluronic acid, dextran sulfate, chondroitin sulfate, dermatan sulfate, mixtures of variably sulfated polysaccharide chains composed of repeating units of D-glucosamine and either L-iduronic or D-glucuronic acids, or salts or derivatives of any of the foregoing.

Also included are negatively charged chitosan derivatives, sodium alginate, chemically-modified dextans, and the like.

III. Preparation of the Composition

A. Liposome Component

Liposomes containing the lipids described above, that is, the neutral cationic lipid and the lipopolymer, can be prepared by a variety of techniques, such as those detailed in Szoka, F., Jr., et al., Ann. Rev. Biophys. Bioeng. 9:467 (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids of the type detailed below are dissolved in a suitable organic solvent which is then evaporated in a vessel to form a thin film. The lipid film is then covered by an aqueous medium, and hydrated to form MLVs, typically with sizes between about 0.1 to 10 microns. The MLVs can then be sonicated if desired to further reduce the size distribution of the liposomes.

Liposomes for use in the composition of the invention include (i) the neutral cationic lipid according to formula (I) and can include additional vesicle forming lipids or a lipid that is stably incorporated into the liposome lipid bilayer, such as diacylglycerols, lyso-phospholipids, fatty acids, glycolipids, cerebrosides and sterols, such as cholesterol. Additional cationic or neutral cationic lipids can be included if desired. A lipopolymer can also be included. In certain preferred embodiments, the hydrophilic polymer is attached through a cleavable linkage.

Typically, liposomes are comprised of between about 5-80 mole percent of the neutral cationic lipid of formula (I), more preferably between about 10-60 mole percent, and still more preferably between about 20-45 mole percent. A lipopolymer is typically included in a molar percentage of between about 1-30, more preferably between about 2-15 mole percent, and still more preferably between about 4-12 mole percent. In studies performed in support of the invention, described below, liposomes comprised of 30 to 60 mole percent neutral cationic lipid and up to 5 mole percent of lipopolymer were utilized.

Liposomes prepared in accordance with the invention can be sized to have substantially homogeneous sizes in a selected size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS-MANUFACTURING AND PRODUCTION TECHNOLOGY, (P. Tyle, Ed.) Marcel Dekker, New York, pp. 267-316 (1990)).

B. Preparation and Characterization of Exemplary Compositions

In studies performed in support of the invention, a pNSL luciferase plasmic DNA with a CMV promoter was entrapped in liposomes comprised of the neutral cationic lipid. In some of the studies, a cleavable lipopolymer was included in the liposome, as described in Zalipsky, S., et al., (2001) “New approach to gene delivery mediated by reversible PEGylation of cationic lipid-DNA complexes,” in Proceed. Intl. Symp. Control. Rel. Bioact. Mater. 28:1177 (#7066). Targeting of the complexes was achieved by including either folate or FGF as targeting ligands. Typically, the targeting ligand was covalently attached to the distal end of the PEG chain of the lipopolymer according to conventional chemistry techniques known in the art and described, for example, in U.S. Pat. No. 6,180,134 and Klibanov, A. L., (2003) “Long-circulating sterically protected liposomes” in Liposomes: A Practical Approach, 2^{nd} Edition, Torchilin, V. P., et al., Eds., Oxford University Press, pp. 231-265.

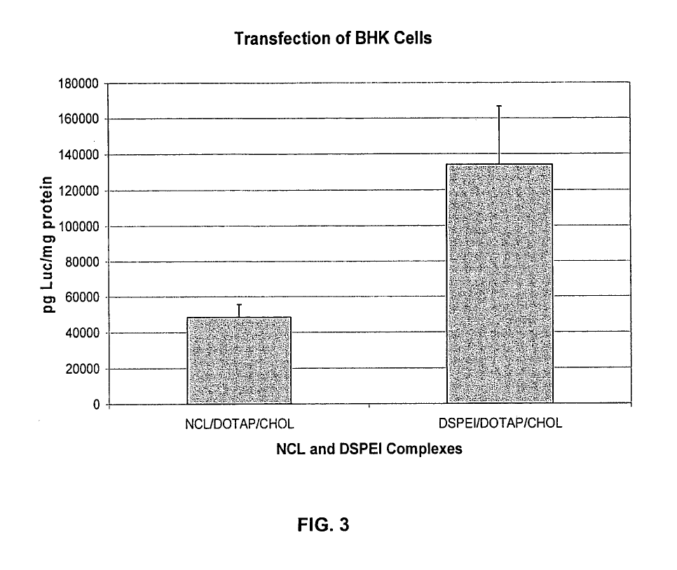

Example 8 illustrates the in vitro transfection and expression of BHK cells using DSPEI liposomes. BHK cells expressing luciferase were identified and gene expression, and hence transfection efficiency, was compared for DSPEI and HDSG containing liposomes. As shown in FIG. 3, much greater gene expression was achieved using DSPEI containing liposomes in comparison with liposomes containing HSDG. The enhancement in gene expression is almost three fold greater using the DSPEI containing liposomes.

Example 9 describes preparation of Formulation Nos. (9-1), (9-2), (9-3), (9-4) and (9-5) for in vivo administration to mice bearing Lewis lung carcinoma cell tumors. Formulation Nos. 2 and 3 included HDSG and the mPEG-DTB-lipid described in U.S. Application Publication No. U.S. 2003/0031764, where R was H (also referred to herein as “FC PEG” or “fast-cleavable” PEG). The formulations also included an FGF targeting ligand. Formulations Nos. 1, 4 and 5 served as comparative controls. The liposome-DNA complexes were administered intravenously to the test mice. Twenty four hours later, tumor and other tissues were collected and analyzed for luciferase expression. The results are shown in Table 1.

TABLE 1
Luciferase Expression in Lewis-lung carcinoma bearing mice after
intravenous administration of FGF-targeted liposome formulations

| Formulation No. | | Luciferase Expression | | |
|---|---|---|---|---|
| (See Example 9 | Targeting | (pg luciferase/mg protein) | | |
| for details) | Ligand | Tumor | Lung | Liver |
| Formulation No. 9-1 | FGF | 15.3 | 1.4 | 1.2 |
| (HDSG/CHOL) | | | | |
| Formulation No. 9-2 | FGF | 7.8 | 1.9 | 4.5 |
| (HDSG/CHOL/F-C PEG) | | | | |
| Formulation No. 9-3 | FGF | 1.2 | 2.0 | 3.2 |
| (HDSG/PHSPC/F-C PEG) | | | | |
| Formulation No. 9-4 | FGF | 3.7 | 2.0 | 4.6 |
| (HDSG/PHSPC/PEG) | | | | |
| Formulation No. 9-5 | folate | 4.3 | 403.9 | 25.4 |
| (DDAB/CHOL) | | | | |

The luciferase expression in the lung for the liposomes composed of DDAB (Formulation No. 9-5), which are cationic liposomes, is nearly 100-fold higher than the other formulations. While the targeting ligand in this formulation differed from the other formulations, the high lung expression for Formulation No. 9-5 is primarily due to the large surface area in the lung and the electrostatic charge interaction between the positively charged plasmid-liposome complexes and the negatively charged endothelial cell surfaces in the lung. The liposome composition where the neutral cationic lipid HDSG is used (Formulation No. 9-1) rather than the cationic lipid DDAB overcomes this problem. Formulations 9-1,9-2, 9-3, and 9-4 all include the HDSG neutral-cationic lipid. Since the lipid is neutral at physiologic pH (7.4) the liposomes do not stick to the lung surfaces, allowing the liposomes to distribute systemically. This improved biodistribution is reflected in the higher luciferase expression in the tumor tissue for Formulations 9-1 and 9-2.

Example 10 describes additional studies, where FGF-targeted liposome/DNA complexes were administered to mice inoculated with Lewis lung tumors and to mice injected with Matrigel, an FGF-angiogenic endothelial cell model for tumor vasculature targeting. In this study, tumor cells and Matrigel were implanted in the same mouse on opposing flanks. Liposomes were prepared composed of the neutral-cationic lipid HDSG and either cholesterol or PHSPC. PEG-DTB-lipid was also included in the formulations in accord with the invention. A cationic lipid was also included in the complexes, to determine the effect of the cationic lipid on complex stability and transfection efficiency. Two cationic lipids were utilized, DOTAP and N²-[N²,N⁵-bis(3-aminopropyl)-L-ormithyl]-N,N-dioctadecyl-L-glutamine tetrahydrotrifluoroacetate, referred to herein as “GC33”.

The formulations were administered intravenously to the tumor-bearing or Matrigel-bearing mice and luciferase expression was measured in the Matrigel or tumor, in the lung, and in the liver 24 hours after administration. The results are shown in Table 2.

TABLE 2

| | | Luciferase Expression (pg | | |
|---|---|---|---|---|
| | Targeting | luciferase/mg protein) | | |
| Formulation No. | Ligand | Matrigel | Lung | Liver |
| Formulation No. 10-1 | none | 28.6 | 2286.1 | 18.1 |
| (DOTAP/Chol) | | | | |
| Formulation No. 10-2 | FGF | 16.0 | 126.7 | 3.1 |
| (HDSG/PHSPC) | | | | |
| Formulation No. 10-3 | FGF | 8.9 | 4.1 | 1.2 |
| (HDSG/PHSPC/FC-PEG) | | | | |
| Formulation No. 10-4 | none | 9.9 | 4.4 | 1.7 |
| (HDSG/DOTAP/PHSPC) | | | | |
| Formulation No. 10-5 | FGF | 10.3 | 3.8 | 1.6 |
| (HDSG/DOTAP/PHSPC) | | | | |
| Formulation No. 10-6 | FGF | 14.2 | 2.0 | 1.3 |
| (HDSG/DOTAP/ | | | | |
| PHSPC/FC-PEG) | | | | |
| Formulation No. 10-7 | none | 10.5 | 223.1 | 2.7 |
| (HDSG/GC33/PHSPC) | | | | |
| Formulation No. 10-8 | FGF | 11.2 | 121.3 | 3.1 |
| (HDSG/GC33/PHSPC) | | | | |
| Formulation No. 10-9 | FGF | 11.3 | 96.0 | 2.2 |
| (HDSG/GC33/PHSPC/FC-PEG) | | | | |

Similarly, Examples 11 and 12 describe in vivo administration of DSPEI containing liposomes, in support of evaluating the in vivo efficacy of the liposomal formulations prepared using the neutral cationic lipid according to formula (I). In comparison with liposomal compositions containing HDSG, the liposomes containing DSPEI are expected to provide a more specific and targeted interaction with the target tumor tissue.

### EXAMPLES

It is to be understood that while the invention has been described in conjunction with the preferred specific embodiments thereof, the foregoing description, as well as the examples that follow, are intended to illustrate and not limit the scope of the invention. Other aspects, advantages and modifications will be apparent to those skilled in the art to which the invention pertains.

All patents, patent applications, journal articles and other references cited herein are incorporated by reference in their entireties.

The following examples are put forth so as to provide those of ordinary skill in the art with a complete disclosure and description of how to make and use the compounds of the invention, and are not intended to limit the scope of what the inventors regard as their invention. Efforts have been made to ensure accuracy with respect to numbers (e.g., amounts, temperatures, etc.) but some errors and deviations should be accounted for. Unless indicated otherwise, parts are parts by weight, temperature is in degrees Celsius (° C.), and pressure is at or near atmospheric.

In the procedures set forth below and throughout this specification, the abbreviations employed have their generally accepted meanings, as follows:
C Celsius (or Centigrade)
mM millimolar
μM micromolar
pmol picomole (10⁻¹² mole)
mg milligram
μg microgram
mL milliliter
μL microliter
μm micrometer
Tm melting temperature
FBS fetal bovine serum
DMEM Dulbeco's Modified Eagle's Medium
DOTAP 1,2-dioleyl-3-trimethylammonium-propane
DSPE distearoylphosphatidylethanolamine
GC33 N²-[N²,N⁵-bis(3-aminopropyl)-L-ormithyl]-N,N-dioctadecyl-L-glutamine tetrahydrotrifluoroacetate;

Materials: The following materials were obtained from the indicated source: partially hydrogenated soy phosphatidylcholine (Vernon Walden Inc., Green Village, N.J.); cholesterol (Solvay Pharmaceuticals, The Netherlands); dioleoylphosphatidyl ethanolamine (DOPE), distearoylphosphatidylethanolamine (DSPE) and dimethyldioctadecylammonium (DDAB) (Avanti Polar Lipids, Inc., Birmingham, Ala.).

Methods Dynamic light scattering was performed using a Coulter N4-MD (Coulter, Miami Fla.).

### Example 1

### Preparation of Exemplary Neutral-Cationic Lipid

### Preparation of Imidazolyl Derivatized Distearoylphosphatidylethanolamine

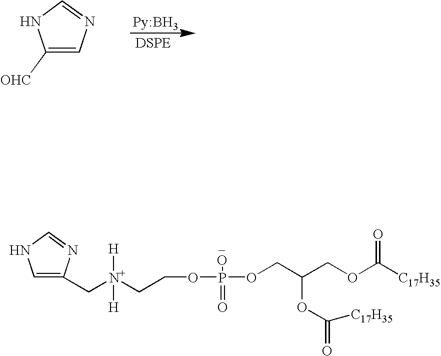

4(5)-Imidazole carboxaldehyde (Aldrich, 0.06 g, 0.6 mmol) and distearoylphosphatidylethanolamine (DSPE) (0.39 g, 0.52 mmol) were dissolved in a mixture of CHCl₃:CH₃OH (1:1 v/v, 16 ml) at 50° C. for 15 min. To the above mixture, borane-pyridine complex (0.05 ml, 0.6 mmol) was added drop wise and the reaction mixture was stirred at 50° C. for 3 hrs and then at room temperature for 18 hrs. The TLC (CHCl₃:CH₃OH: H₂O, 80:18:2) of reaction mixture showed that the reaction went to completion. The solvent was evaporated and the crude mixture obtained was chromatographed using silica gel. CHCl₃:CH₃OH (80:18) was used as an eluent to remove upper impurities followed by CHCl₃:CH₃OH:H₂O (80:18:2) solvent system to elute the white solid product which was lyophilized from tertiary butanol. The yield of product was 0.37 g, (86%). ¹H NMR (CDCl₃): δ 0.878 (t, 6H, CH₃), 1.25-1.75 (m, 48H, lipid CH₂), 1.59 (m, 4H, lipid CH₂), 2.30 (m, 4H, CO—CH₂), 2.74 (m, 2H, NH₂—CH₂), 3.67 (m, 2H, CH₂—NH₂), 4.02 (m, 2H, CH₂—OPO₃), 4.22 (m, 2H, OPO₃—CH₂), 4.42 (d, 2H, CH₂—O—CO), 5.27 (m, 1H, CH₂—CH—CH₂), 6.87 (s, 1H, N—CH—C), 7.54 (m, 1H, N—CH—NH) ppm. ¹³C NMR (CDCl₃): δ 14.11, 22.67, 24.88, 29.12, 29.16, 29.35, 29.53, 29.66, 29.71, 31.90, 34.13, 34.29, 49.48, 55.71, 62.58, 63.46, 64.22, 70.14, 70.20, 119.97, 128.81, 130.88, 131.01, 134.17, 173.09, 173.48 ppm.

### Example 2

### Preparation of Diimidazole Phosphatidylethanolamine

The same procedure was utilized as described in Example 1, with double the amount of imidazole carboxaldehyde (1 mmole) and borane-pyridine (1.1 mmole), to produce the titled derivative. The di-imidazole product was purified by chromatography on silica gel and characterized by MALDI-TOF mass spectrometry. The product had a molecular weight of 907 g/mol indicative of two imidazole moieties attached to the quaternary amine of phosphatidylethanolamine. This reaction is also depicted schematically in FIG. 1. The same ¹H NMR spectrum was seen as described in Example 1, with integration confirming the presence of two imidazole moieties.

### Example 3

### Preparation of Liposomes Containing DSPEI and PHSPC

DSPEI and PHSPC were mixed at the molar ratio of 40:60 and were dissolved in chloroform. Chloroform was evaporated with rotary evaporation in order to form a lipid thin film. Lipid thin film was hydrated with pH ˜4.5 water for 30 min at ˜40° C. The resulted multi-layer liposomes were sonicated for ˜10 min, and final liposome size was around 80 nm.

### Example 4

### Preparation of Liposomes Containing DSPEI, DOTAP and Cholesterol

DSPEI, DOTAP and CHOL were mixed at the molar ratio of 35:30:35 (molar ratio) and were dissolved in chloroform. Chloroform was evaporated with rotary evaporation in order to form a lipid thin film. Then the lipid thin film was hydrated with pH 3-3.5 water for 30 min at ˜40° C. The resulted multi-layer liposomes were sonicated for ˜20 min, and final liposome size was around 100 nm.

### Preparation of DSPEI Liposomes

| Formulation | pH | Hydration | Sonication | Size (nm) |
|---|---|---|---|---|
| DSPEI/PHSPC | 4.5 | easy | 10 min | 80 |
| (40:60) | | | | |
| DSPEI/DOTAP/CHOL | 3 | easy | 30 min | 100 |
| (35:30:35) | | | | |

### Example 5

### Zeta Potential Determination for Liposomes Containing Neutral Cationic Lipid

Zeta potential was measured using a ZETASIZER 2000 from Malver Instruments, Inc. (Southborough Mass.). The instrument was operated as follows: number of measurements: 3; delay between measurements: 5 seconds; temperature: 25° C.; viscosity: 0.89 cP; dielectric constant: 79; cell type: capillary flow; zeta limits: −150 mV to 150 mV. Zeta potential measurements were obtained from liposomes containing DSPEI and PHSPC, prepared as described in Example 3, and on comparative liposomes comprised of HDSG and of DDAB. The results are shown in FIG. 2.

### Example 6

### Preparation of Liposomes Containing Nucleic Acid

Liposomes containing DSPEI were prepared as described in Examples 3 and 4 above. Liposomes containing the neutral cationic lipid HDSG were prepared by preparing a solution of the desired lipid components in an organic solvent in the desired molar ratio and then hydrated with 5% glucose, pH 4 to 5. The lipid components and the mole ratio of the components are specified in the Examples below.

A pNSL plasmid encoding for luciferase was constructed as described in U.S. Pat. No. 5,851,818 from two commercially available plasmids, pGFP-N1 plasmid (Clontech, Palo Alto, Calif.) and pGL3-C (Promega Corporation, Madison, Wis.). DNA-liposome complexes were prepared by transferring the plasmid carrying luciferase gene to liposomes, composed of DSPEI or HDSG, DOTAP and cholesterol at a ratio of 1 μg DNA to 14 mmole total lipids. The luciferase reporter plasmid DNA solution was added to the acidic liposome solution slowly with continuous stirring for 10 minutes.

### Example 7

### Preparation of DNA-Liposomes Containing Targeting Ligands

FGF or folate ligands were conjugated to maleimide-PEG-DSPE (mPEG-DSPE), according to procedures known in the art (Gabizon, A. et al, Bioconjugate Chem., 10:289 (1999)).

Liposomes were prepared as described in Examples 3 and 4. DNA-liposome complexes were incubated with micellar solutions of mPEG-DSPE, FGF-PEG-DSPE or folate-PEG-DSPE for 2-3 hours to achieve insertion of the ligand-PEG-lipid into the pre-formed liposomes.

### Example 8

### In Vitro Transfection and Expression Using DSPEI and HDSG Liposomes

Baby hamster kidney (BHK) cells were seeded on 6-well plates, at ˜1×10⁴ cells/well, and incubated for 2 days. Then BHK cells were transfected with DNA-liposome complexes prepared as described in Example 6 using either DSPEI-containing liposomes or HDSG-containing liposomes, at 1 μg of plasmid DNA/well, by incubating the cells in the presence of the DNA-liposome complexes for 5 hrs, followed by replacing the DNA-Liposome complexes, with regular media. Cells were harvested after 20 hrs and assayed for expression of the reporter gene, luciferase, which was presented as picogram luciferase/mg protein. The results are shown in FIG. 3.

### Example 9

### In Vivo Transfection and Expression in Tumor Tissue Using HDSG-Liposomes and FGF- or Folate Targeting Ligand

A. Tumor Models

KB tumor cells (1 million cells) were inoculated subcutaneously to the flank of nude mice. The mice were fed a reduced folate diet to upregulate the expression of folate receptors on the KB tumor cells. This model was used for folate-conjugated liposome-DNA complexes to target tumor vasculature angiogenic endothelial cells.

Lewis lung carcinoma cells (1 million cells) were inoculated subcutaneously to the flank of B6C3-F1 mice. FGF receptors were expressed either on the surface of angiogenic endothelial cells or tumor cells. This model was used for FGF-conjugated liposome-DNA complexes to target tumor vasculature angiogenic endothelial cells.

B. Liposome Formulations

Five liposome formulations were prepared as described in Example 6 with the following lipid components:

### Formulation No. 9-1

| Component | Amount | |
|---|---|---|
| HDSG Neutral-cationic lipid | 60 | mole percent of total lipids |
| Cholesterol | 40 | mole percent of total lipids |
| luciferase plasmid | 100 | μg |
| FGF targeting ligand | 15 | FGF/liposome |

### Formulation No. 9-2

| Component | Amount | |
|---|---|---|
| HDSG Neutral-cationic lipid | 60 | mole percent of total lipids |
| cholesterol | 40 | mole percent of total lipids |
| mPEG-DTB-DSPE (“FC PEG) | 5 | mole percent of total lipids |
| luciferase plasmid | 100 | μg |
| FGF targeting ligand | 15 | FGF/liposome |

### Formulation No. 9-3

| Component | Amount | |
|---|---|---|
| HDSG Neutral-cationic lipid | 40 | mole percent of total lipids |
| PHSPC | 60 | mole percent of total lipids |
| mPEG-DTB-DSPE (“FC PEG) | 5 | mole percent of total lipids |
| luciferase plasmid | 100 | μg |
| FGF targeting ligand | 15 | FGF/liposome |

### Formulation No. 9-4

| Component | Amount | |
|---|---|---|
| HDSG Neutral-cationic lipid | 40 | mole percent of total lipids |
| PHSPC | 60 | mole percent of total lipids |
| mPEG-DSPE | 5 | mole percent of total lipids |
| luciferase plasmid | 100 | μg |
| FGF targeting ligand | 15 | FGF/liposome |

### Formulation No. 9-5

| Component | Amount | |
|---|---|---|
| DDAB | 55 | mole percent of total lipids |
| PHSPC | 45 | mole percent of total lipids |
| luciferase plasmid | 100 | μg |
| folate targeting ligand | 15 | FGF/liposome |

C. In Vivo Administration

Fifteen test mice injected with Lewis lung carcinoma cells were randomly divided into four test groups to receive one of Formulations 1-5. The liposome-DNA complexes were administered intravenously at a dose of 200 μg DNA plasmid. Tumor and other tissues were collected 24 hours after treatment and luciferase expression was determined by luciferase assay from the tissue extracts. The results are shown in Table 1.

### Example 10

### In Vivo Administration of FGF-Targeted HDSG-Liposome-DNA Complexes

A. Matrigel Tumor Model

A Matrigel® model in mice was employed for tumor vasculature targeting of FGF-angiogenic endothelial cells. Angiogenic endothelial cells in Matrigel® are similar to vasculature angiogenic endothelial cells in tumor, these endothelial cells (endothelial cells only, without tumor cells) in Matrigel® were used to mimic endothelial cells in tumor for the study of in vivo FGF-targeted liposome/nucleic acid complex transfection and expression. Matrigel® forms a solid gel when injected into mice subcutaneously and induces a rapid and intense angiogenic reaction.

B. Liposome Formuations

Nine liposome formulations were prepared as described in Example 6 with the following lipid components:

### Formulation No. 10-1

| Component | Amount | |
|---|---|---|
| DOTAP | 55 | mole percent of total lipids |
| cholesterol | 45 | mole percent of total lipids |
| luciferase plasmid | 100 | μg |

### Formulation No. 10-2

| Component | Amount | |
|---|---|---|
| HDSG Neutral-cationic lipid | 40 | mole percent of total lipids |
| PHSPC | 60 | mole percent of total lipids |
| luciferase plasmid | 200 | μg |
| FGF targeting ligand | 15 | FGF/liposome |

### Formulation No. 10-3

| Component | Amount | |
|---|---|---|
| HDSG Neutral-cationic lipid | 40 | mole percent of total lipids |
| PHSPC | 60 | mole percent of total lipids |
| FC-PEG | 1 | mole percent of total lipids |
| luciferase plasmid | 200 | μg |
| FGF targeting ligand | 15 | FGF/liposome |

### Formulation No. 10-4

| Component | Amount | |
|---|---|---|
| HDSG Neutral-cationic lipid | 35 | mole percent of total lipids |
| DOTAP | 30 | mole percent of total lipids |
| PHSPC | 35 | mole percent of total lipids |
| luciferase plasmid | 200 | μg |

### Formulation No. 10-5

| Component | Amount | |
|---|---|---|
| HDSG Neutral-cationic lipid | 35 | mole percent of total lipids |
| DOTAP | 30 | mole percent of total lipids |
| PHSPC | 35 | mole percent of total lipids |
| luciferase plasmid | 200 | μg |
| FGF targeting ligand | 15 | FGF/liposome |

### Formulation No. 10-6

| Component | Amount | |
|---|---|---|
| HDSG Neutral-cationic lipid | 35 | mole percent of total lipids |
| DOTAP | 30 | mole percent of total lipids |
| PHSPC | 35 | mole percent of total lipids |
| FC-PEG | 1 | mole percent of total lipids |
| luciferase plasmid | 200 | μg |
| FGF targeting ligand | 15 | FGF/liposome |

### Formulation No. 10-7

| Component | Amount | |
|---|---|---|
| HDSG Neutral-cationic lipid | 42.5 | mole percent of total lipids |
| GC33 | 22.5 | Mole percent of total lipids |
| PHSPC | 35 | Mole percent of total lipids |
| luciferase plasmid | 250 | μg |

### Formulation No. 10-8

| Component | Amount | |
|---|---|---|
| HDSG Neutral-cationic lipid | 42.5 | mole percent of total lipids |
| GC33 | 22.5 | mole percent of total lipids |
| PHSPC | 35 | mole percent of total lipids |
| luciferase plasmid | 250 | μg |
| FGF targeting ligand | 15 | FGF/liposome |

### Formulation No. 10-9

| Component | Amount | |
|---|---|---|
| HDSG Neutral-cationic lipid | 42.5 | mole percent of total lipids |
| GC33 | 22.5 | mole percent of total lipids |
| PHSPC | 35 | mole percent of total lipids |
| FC-PEG | 1 | mole percent of total lipids |
| luciferase plasmid | 250 | μg |
| FGF targeting ligand | 15 | FGF/liposome |

C. In Vivo Administration

Twenty-seven mice were injected with Matrigel. Six days after implantation of the Matrigel, the mice were randomized into treatment groups (n=3) for treatment with one of nine formulations described in section B above. The liposome-DNA complexes were administered intravenously at a dose of 200 μg DNA plasmid. Twenty-four hours after administration of the FGF-targeted liposome-DNA complexes, luciferase expression in the matrigel, lung and liver was measured. The results are shown in Table 2.

### Example 11

### In Vivo Administration of FGF-Targeted DSPEI-Liposome-DNA Complexes

A. Test Animals

Mice are inoculated with Lewis lung carcinoma cells as described in Example 9A.

B. Liposome Formuations

Nine liposome formulations are prepared as described in Examples 6 and 7 with the following lipid components:

### Formulation No. 11-1

| Component | Amount | |
|---|---|---|
| DOTAP | 55 | mole percent of total lipids |
| cholesterol | 45 | mole percent of total lipids |
| luciferase plasmid | 100 | μg |

### Formulation No. 11-2

| Component | Amount | |
|---|---|---|
| DSPEI Neutral cationic lipid | 40 | mole percent of total lipids |
| PHSPC | 60 | mole percent of total lipids |
| luciferase plasmid | 200 | μg |
| FGF targeting ligand | 15 | FGF/liposome |

### Formulation No. 11-3

| Component | Amount | |
|---|---|---|
| DSPEI Neutral cationic lipid | 40 | mole percent of total lipids |
| PHSPC | 60 | mole percent of total lipids |
| FC-PEG | 1 | mole percent of total lipids |
| luciferase plasmid | 200 | μg |
| FGF targeting ligand | 15 | FGF/liposome |

### Formulation No. 11-4

| Component | Amount | |
|---|---|---|
| DSPEI Neutral cationic lipid | 35 | mole percent of total lipids |
| DOTAP | 30 | mole percent of total lipids |
| PHSPC | 35 | mole percent of total lipids |
| luciferase plasmid | 200 | μg |

### Formulation No. 11-5

| Component | Amount | |
|---|---|---|
| DSPEI Neutral cationic lipid | 35 | mole percent of total lipids |
| DOTAP | 30 | mole percent of total lipids |
| PHSPC | 35 | mole percent of total lipids |
| luciferase plasmid | 200 | μg |
| FGF targeting ligand | 15 | FGF/liposome |

### Formulation No. 11-6

| Component | Amount | |
|---|---|---|
| DSPEI Neutral cationic lipid | 35 | mole percent of total lipids |
| DOTAP | 30 | mole percent of total lipids |
| PHSPC | 35 | mole percent of total lipids |
| FC-PEG | 1 | mole percent of total lipids |
| luciferase plasmid | 200 | μg |
| FGF targeting ligand | 15 | FGF/liposome |

### Formulation No. 11-7

| Component | Amount | |
|---|---|---|
| DSPEI Neutral cationic lipid | 42.5 | mole percent of total lipids |
| GC33 | 22.5 | Mole percent of total lipids |
| PHSPC | 35 | Mole percent of total lipids |
| luciferase plasmid | 250 | μg |

### Formulation No. 11-8

| Component | Amount | |
|---|---|---|
| DSPEI Neutral cationic lipid | 42.5 | mole percent of total lipids |
| GC33 | 22.5 | mole percent of total lipids |
| PHSPC | 35 | mole percent of total lipids |
| luciferase plasmid | 250 | μg |
| FGF targeting ligand | 15 | FGF/liposome |

### Formulation No. 11-9

| Component | Amount | |
|---|---|---|
| DSPEI Neutral cationic lipid | 42.5 | mole percent of total lipids |
| GC33 | 22.5 | mole percent of total lipids |
| PHSPC | 35 | mole percent of total lipids |
| FC-PEG | 1 | mole percent of total lipids |
| luciferase plasmid | 250 | μg |
| FGF targeting ligand | 15 | FGF/liposome |

C. In Vivo Administration

Nine-days after inoculation with tumor cells, twenty-seven tumor-bearing mice are randomized into treatment groups (n=3) for treatment with one of nine formulations, Formulation No. (11-1) through Formulation No. (11-9). The liposome-DNA complexes are administered intravenously at a dose of 200 μg DNA plasmid. Twenty-four hours after administration of the FGF-targeted liposome-DNA complexes, luciferase expression in the tumor, lung and liver is measured.

### Example 12

### In Vivo Administration of FGF-Targeted Liposome-DNA Complexes

A. Test Animals

Mice are inoculated with Lewis lung carcinoma cells as described in Example 9A. On the opposing flank, Matrigel is injected as described in Example 10A.

B. Liposome Formulations

Seven liposome formulations are prepared as described in Examples 6 and 7 with the following lipid components:

### Formulation No. 12-1

| Component | Amount | |
|---|---|---|
| DOTAP | 55 | mole percent of total lipids |
| cholesterol | 45 | mole percent of total lipids |
| luciferase plasmid | 100 | μg |

### Formulation No. 12-2

| Component | Amount | |
|---|---|---|
| DSPEI Neutral cationic lipid | 35 | mole percent of total lipids |
| DOTAP | 30 | mole percent of total lipids |
| CHOL | 35 | mole percent of total lipids |
| luciferase plasmid | 200 | μg |

### Formulation No. 12-3

| Component | Amount | |
|---|---|---|
| DSPEI Neutral cationic lipid | 35 | mole percent of total lipids |
| DOTAP | 30 | mole percent of total lipids |
| CHOL | 35 | mole percent of total lipids |
| luciferase plasmid | 200 | μg |
| FGF targeting ligand | 15 | FGF/liposome |

### Formulation No. 12-4

| Component | Amount | |
|---|---|---|
| DSPEI Neutral cationic lipid | 35 | mole percent of total lipids |
| DOTAP | 30 | mole percent of total lipids |
| CHOL | 35 | mole percent of total lipids |
| FC-PEG | 1 | mole percent of total lipids |
| luciferase plasmid | 200 | μg |
| FGF targeting ligand | 15 | FGF/liposome |

Formulation No. 12-5

| Component | Amount |
|---|---|
| DSPEI Neutral cationic lipid | 53.75 mole percent of total lipids |
| GC33 | 11.25 mole percent of total lipids |
| PHSPC | 35 mole percent of total lipids |
| luciferase plasmid | 200 μg |

Formulation No. 12-6

| Component | Amount |
|---|---|
| DSPEI Neutral cationic lipid | 53.75 mole percent of total lipids |
| GC33 | 11.25 mole percent of total lipids |
| PHSPC | 35 mole percent of total lipids |
| luciferase plasmid | 200 μg |
| FGF targeting ligand | 15 FGF/liposome |

Formulation No. 12-7

| Component | Amount |
|---|---|
| DSPEI Neutral cationic lipid | 53.75 mole percent of total lipids |
| GC33 | 11.25 mole percent of total lipids |
| PHSPC | 35 mole percent of total lipids |
| FC-PEG | 1 mole percent of total lipids |
| luciferase plasmid | 200 μg |
| FGF targeting ligand | 15 FGF/liposome |

C. In Vivo Administration

Nine-days after inoculation with tumor cells, 21 tumor-bearing mice are randomized into treatment groups (n=3) for treatment with one of formulations, Formulation No. (12-1) through Formulation No. (12-7). The liposome-DNA complexes are administered intravenously at a dose of 200 μg DNA plasmid. Twenty-four hours after administration of the FGF-targeted liposome-DNA complexes, luciferase expression in the matrigel, tumor, lung and liver is measured.

Although the invention has been described with respect to particular embodiments, it will be apparent to those skilled in the art that various changes and modifications can be made without departing from the invention.

1. A compound according to formula (I) 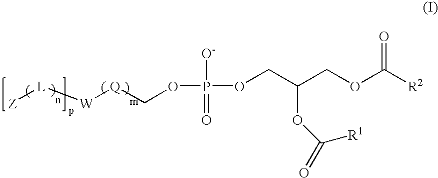
wherein each of R¹ and R² is independently selected from H or a branched or unbranched alkyl, alkenyl, or alkynyl chain having between 6-24 carbon atoms;
n=1-20;
m=1-20;
p=1-3;
L and Q are independently selected from the group consisting of C₁-C₆ alkyl, —X—(C═O)—Y—CH₂—, —X—(C═O)—, —X—CH₂—, where X and Y are independently selected from oxygen, NH and a direct bond;
W is an amino, guanidino or amidino moiety; and
Z is a weakly basic moiety that has a pKₐ of less than 7.4 and greater than about 4.0.

2. The compound of claim 1, wherein p is 1 and W is —NR⁸²—, wherein each R⁸ is independently selected from H or C₁₋₆ alkyl.

3. The compound of claim 1, wherein p is 2 and W is —NR⁸—, wherein R⁸ is H or C₁₋₆ alkyl.

4. The compound of claim 1, wherein Z is a cyclic or acyclic amine.

5. The compound of claim 1, wherein Z is imidazole.

6. The compound of claim 1, wherein each of R¹ and R² is C₁₇H₃₅.

7. A composition, comprising:
liposomes comprising a neutral cationic lipid according to claim 1 and a polyanionic compound.

8. The composition of claim 7, wherein the polyanionic compound is a polynucleotide, a polysaccharide or a negatively charged protein.

9. The composition of claim 8, wherein the polynucleotide is a plasmid, DNA, RNA, a DNA/RNA hybrid, an oligonucleotide, an antisense oligonucleotide, a small interfering RNA, a protein-nucleic acid complex, a polynucleotide-drug conjugate, or mixtures thereof.

10. The composition of claim 8, wherein the polynucleotide comprises a modified nucleotide, a non-naturally occurring nucleotide, a polynucleotide analog having surrogate linkers, a hybrid polynucleotide comprising pentavalent phosphate linkers and surrogate linkers, or mixtures thereof.

11. The composition of claim 7, further comprising a lipopolymer.

12. The composition of claim 11, wherein said lipopolymer is comprised of a hydrophilic polymer selected from the group consisting of polyethyleneglycol, polyvinylpyrrolidone, polyvinylmethylether, polyhydroxypropyl methacrylate, polyhydroxyethyl methacrylate, polyhydroxyethyl acrylate, polymethacrylamide, poly-dimethylacrylamide, polymethyloxazoline, polyethyloxazoline, polyhydroxyproploxazoline, polyaspartamide, and polyethyleneoxide-polypropylene oxide, copolymers thereof and mixtures thereof.

13. The composition of claim 12, wherein the hydrophilic polymer is attached to a lipid moiety of the lipopolymer via a cleavable linkage.

14. The composition of claim 7, wherein said liposomes comprise between 5-80 mole percent of the lipid of formula I.

15. The composition of claim 11, wherein said liposomes comprise between about 1-30 mole percent of the lipopolymer.

16. The composition of claim 7, further including a therapeutic agent entrapped in the liposomes.

17. The composition of claim 7, wherein said polyanionic compound is entrapped in at least a portion of said liposomes.

18. The composition of claim 7, further comprising a targeting ligand for targeting the liposomes to a target site.

19. The composition of claim 18, wherein the targeting ligand has binding affinity for endothelial cells or tumor cells.

20. The composition of claim 19, wherein said targeting ligand is a c-erbB-2 protein product of the HER2/neu oncogene, epidermal growth factor (EGF), basic fibroblast growth (basic FGF), vascular endothelial growth factor, E-selectin, L-selectin, P-selectin, folate, CD4, CD19, αβ integrin, or a chemokine.

21. A method of preparing liposomes for administration of a polyanionic compound characterized by an extended blood circulation time, comprising
forming liposomes from vesicle-forming lipids comprising a neutral cationic lipid having a structure according to formula (I) of claim 1
adding a polyanionic compound, and
sizing the liposomes to a selected size in the size range between about 0.05 to 0.5 microns.

22. The method of claim 21, wherein the liposomes further comprise a therapeutic agent in entrapped form.

23. A method of transfecting a cell, comprising contacting a cell with the composition of claim 7.

24. A composition for administration of a polyanionic compound, comprising:
liposomes comprising
(i) a neutral cationic lipid having a structure according to formula (I) 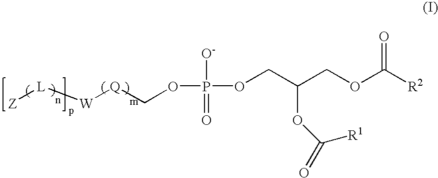
wherein each of R¹ and R² is a branched or unbranched alkyl, alkenyl, or alkynyl chain having between 6-24 carbon atoms;
n=1;
m=1;
p=1;
L and Q are independently selected from the group consisting of C₁-C₆ alkyl; W is —NR⁸²—, wherein each R⁸ is independently selected from H or C₁₋₆ alkyl;
Z is imidazole; and
(ii) at least one of a plasmid, a DNA, an RNA, a DNA/RNA hybrid, an oligonucleotide, an antisense oligonucleotide, a small interfering RNA, a polynucleotide analog having surrogate linkers; or a hybrid polynucleotide comprising pentavalent phosphate linkers and surrogate linkers, and
(iii) a lipopolymer or a targeting ligand.
